(19) United States
(12) Patent Application Publication
(10) Pub. No.: US 2008/0132834 A1
(43) Pub. Date: Jun. 5, 2008

(54) FLEXIBLE ENDOSCOPE TIP BENDING MECHANISM USING OPTICAL FIBERS AS TENSION MEMBERS

(75) Inventor: Charles David Melville, Issaquah, WA (US)

Correspondence Address: LAW OFFICES OF RONALD M ANDERSON, 600 108TH AVE, NE, SUITE 507, BELLEVUE, WA, 98004, US

(73) Assignee: University of Washington, Seattle, WA (US)

(21) Appl. No.: 12/025,342

(22) Filed: Feb. 4, 2008

Related U.S. Application Data
(63) Continuation-in-part of application No. 11/566,597, filed on Dec. 4, 2006.

(51) Int. Cl. A61B 1/07; A61B 1/005; A61B 1/01

(52) U.S. Cl. 604/95.04

(57) ABSTRACT

A flexible endoscope includes an elongate flexible extrusion. A central lumen extends longitudinally through the center of the extrusion, and a plurality of other longitudinally extending lumens are spaced around the central lumen. An optical fiber disposed in the central lumen serves as a compression member and conveys light through the flexible endoscope. One or more pairs of optical fibers disposed around the central lumen convey light and serve as tension members. Applying a tension force to one of these optical fibers causes a distal tip of the flexible endoscope to bend in a first direction, while applying a tension force to the opposite optical fiber causes the distal tip to bend in an opposite direction. The second pair of optical fibers comprising tension members are orthogonally disposed relative to the first pair, and the first and second pair control tip bending in orthogonal directions.

This application is a continuation-in-part of a copending patent application Ser. No. 11/566,597, filed on Dec. 4, 2006, the benefit of the filing date of which is hereby claimed under 35 U.S.C. § 120.

### BACKGROUND

Flexible endoscopes have become increasingly preferred as the instrument of choice for performing certain types of surgical procedures, performing certain diagnostic procedures, or rendering therapy to internal sites within a patient's body. Since the endoscope can be inserted through a natural body opening or through a relatively small transcutaneous incision and advanced to the site where the medical procedure is to be performed, the use of an endoscope exposes the patient to much less trauma and risk of infection than a conventional surgical technique that would otherwise be required to access the internal site. The use of a flexible endoscope is so minimally invasive that some medical procedures can be done in a clinic with the device, and the patient released within an hour or two after a procedure has been completed.

Depending upon the internal site where a medical procedure will be performed with a flexible endoscope, it may be necessary to remotely bend the distal tip of the endoscope, for example, to facilitate advancing the endoscope through a curving body lumen or to position the distal tip at a desired orientation to implement the medical procedure with the endoscope. Although the term “flexible” endoscope indicates that the endoscope is not rigid, some mechanism must be provided to actually bend the flexible distal portion of the endoscope in a desired direction and by a required amount. One approach for bending the distal tip would use wires that extend coaxially along the flexible endoscope, so that when tension is applied to the proximal end of one of the wires relative to the central shaft of the flexible endoscope, the distal end bends toward the side of the central shaft on which the wire is attached. A wire running down the opposite side of the flexible endoscope can be pulled to apply a tension to straighten the distal tip or bend it in the opposite direction. If four of these coaxial wires extend down the length of the flexible endoscope to enable bending of its distal tip in each direction defined by orthogonal X and Y axes, the distal tip can be bent in any desired direction. However, these four tension wires add substantially to the diameter of the device. There is a significant advantage in using a flexible endoscope having a diameter on the order of 1 mm or less. But such a small diameter flexible endoscope can generally not be achieved if the conventional coaxial bending wire arrangement is used to bend the distal tip. Accordingly, a different approach is required that is usable in a flexible endoscope having the desired small diameter, which enables the distal tip to be selectively bent in at least two opposite directions, or better, relative to two orthogonal axes.

### SUMMARY

To enable new endoscopic devices of smaller size to perform the same functions as more conventional endoscopes, it is important to recognize that elements of their design must have multiple functions. In the case of a small flexible endoscope, an exemplary design using an optical fiber as the main light conduit from the proximal end to the distal tip can be used. One interesting mechanical property of an optical fiber, which is made of fused silica, is its mechanical strength. The compressive strength of stainless steel is about 95,000 PSI. In contrast, fused silica has a compressive strength of about 160,000 PSI, which is about 1.6 times the compressive strength of stainless steel. A 125-micron diameter optical fiber has over 3 pounds of compressive strength, which is more than enough to serve as a compressive member for enabling the distal tip bending function required in one exemplary embodiment. A coaxial, “inside out design” is employed to complete the device and uses additional optical fibers that convey light to also serve as tension members, thereby avoiding the need for wires to be provided for this purpose.

More specifically, one exemplary embodiment of a flexible endoscope having a distal tip that can be selectively bent includes a flexible extrusion that is elongate, extending between a proximal end and a distal end. A plurality of lumens are formed within the flexible extrusion. At least two lumens of the plurality of lumens are disposed on opposite sides of, and radially outward of a central lumen within the flexible extrusion. At least a pair of tension members each extend proximally through a different one of the plurality of lumens from an attachment point where the tension member is connected to the flexible extrusion. Each attachment point is disposed adjacent to the distal tip of the flexible extrusion. The tension members are free to slide longitudinally within the lumens proximal of the attachment points. A compression member is disposed within the central lumen and is bonded to the flexible extrusion. When tension is applied to an optical fiber comprising a first tension member relative to the compression member, the distal tip of the flexible endoscope is bent in a first direction. Similarly, when tension is applied to another optical fiber comprising a second tension member that is disposed generally diametrically opposite the first tension member, the flexible endoscope is bent in a second direction that is opposite to the first direction. The distal tip of the flexible endoscope can thus be selectively caused to bend in at least two opposite directions.

In one exemplary embodiment, the compression member comprises a scanning optical fiber. This scanning optical fiber serves a dual purpose, since it also is used for conveying light employed to produce an image of a surface adjacent to a distal end of the flexible extrusion.

If two pairs of lumens are used for conveying tension members and are disposed at cardinal locations around the central lumen, tension can be applied to selected tension members to cause the distal tip to bend relative to two orthogonal axes. The flexible extrusion can be formed of a material selected for a characteristic low coefficient of friction, such as TEFLON™ (i.e., polytetrafluoroethylene), so that the optical fibers comprising the tension fibers can readily slide within the lumens through which they pass.

In at least some embodiments, the plurality of lumens extend helically around the central lumen. By passing the optical fibers comprising the tension member through such helical lumens, the relative length of the optical fibers on opposite sides of the central lumen (i.e., on an inside of a bend and on the outside of the bend) remains generally the same when the distal tip is bent.

From just behind the distal tip to the proximal end of the flexible enclosure, the lumens through which the tension members pass and which are disposed on opposite sides of the central lumen can be diametrically closer together than they are proximate to the distal tip. The greater spacing between the tension members and the compression member at the distal tip provides a greater moment arm for bending the distal tip.

Another aspect of this invention is directed to a method for enabling a distal tip of a flexible endoscope to be selectively bent. The method includes steps that are generally consistent with the functions of the elements of the flexible endoscope discussed above.

This Summary has been provided to introduce a few concepts in a simplified form that are further described in detail below in the Description. However, this Summary is not intended to identify key or essential features of the claimed subject matter, nor is it intended to be used as an aid in determining the scope of the claimed subject matter.

### DRAWINGS

Various aspects and attendant advantages of one or more exemplary embodiments and modifications thereto will become more readily appreciated as the same becomes better understood by reference to the following detailed description, when taken in conjunction with the accompanying drawings, wherein:

FIGS. 1A and 1B are schematic illustrations of two members, showing how the effective relative path lengths of the members change as they are bent from a parallel configuration (FIG. 1A) to a bent configuration (FIG. 1B);

FIG. 2 is a schematic representation of two members, including a helically coiled tension member and an internal compression member;

FIG. 3 is a schematic representation of the two members of FIG. 2, illustrating how bending the two members does not generally change their relative lengths;

FIG. 4 is a cross-sectional perspective view of a portion of an extrusion having a plurality of helical lumens distributed about a central lumen, including two lumens through which tension members comprising optical fibers extend helically and longitudinally, for use in a small diameter flexible endoscope having a selectively bendable distal tip;

FIG. 5 is a cross-sectional perspective view of a portion of an extrusion of FIG. 4 that is actively bent and which has the two lumens spaced near the outer peripheral surface of the extrusion (in the illustrated portion of this exemplary embodiment, the lumens do not wrap around the extrusion and are not bonded to the tension members);

FIG. 6 illustrates a portion of an exemplary embodiment like that of FIG. 5, except that another pair of lumens are provided for tension members to enable the distal tip of the flexible endoscope to be bent in four different directions around two orthogonal axes instead of only in two opposite directions around a single axis;

FIG. 7 is a schematic cross-sectional view of a flexible endoscope with a scanning optical fiber and helically extending tension members comprising optical fibers (only two shown); and

FIG. 8 is a schematic view of an internal lumen/body cavity in which the flexible endoscope of FIG. 7 is illustrated, showing how the distal tip of the flexible endoscope is bent in a desired direction.

### DESCRIPTION

### Figures and Disclosed Embodiments are Not Limiting

Exemplary embodiments are illustrated in referenced Figures of the drawings. It is intended that the embodiments and Figures disclosed herein are to be considered illustrative rather than restrictive. No limitation on the scope of the technology and of the claims that follow is to be imputed to the examples shown in the drawings and discussed herein.

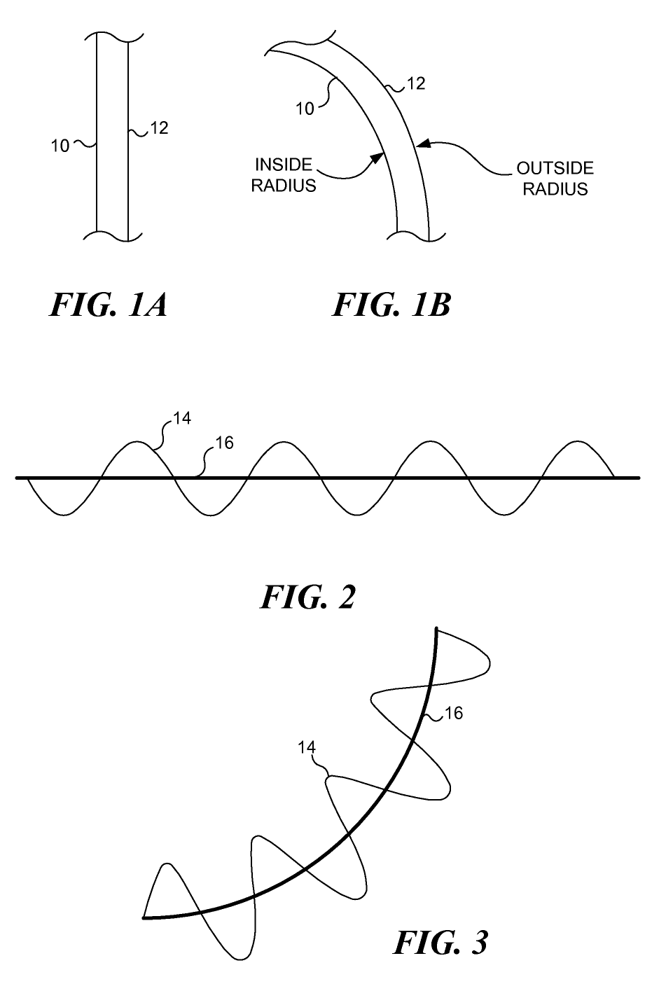

To minimize accidental tip bending while transmitting the compression and tension forces to the distal end, the members' relative path lengths should not change when sections along the length of the flexible endoscope are bent. FIGS. 1A and 1B are schematic drawings illustrating that when two parallel members 10 and 12 are bent, the radius of curvature is less for the inside member than the outside member. Since the path length for the inside member around a curve is less than for the outside member, the inside member should become relatively longer when bent into the curve, compared to the outside member. Accordingly, it can be seen in FIG. 1B that the effective path lengths of the two members, relative to each other, have changed, as a result of the bending of the two members.

To avoid the change in path length when such members are bent requires a modification to the parallel member configuration illustrated in FIG. 1A. Specifically, if a member 14 is wrapped around a member 16 in a helix, as shown in FIG. 2, then when both members are bent, as indicated in FIG. 3, the average length of both members will remain almost the same. Thus, the helical coiling of one member around another member when bending the distal tip of a flexible endoscope can provide the same benefit.

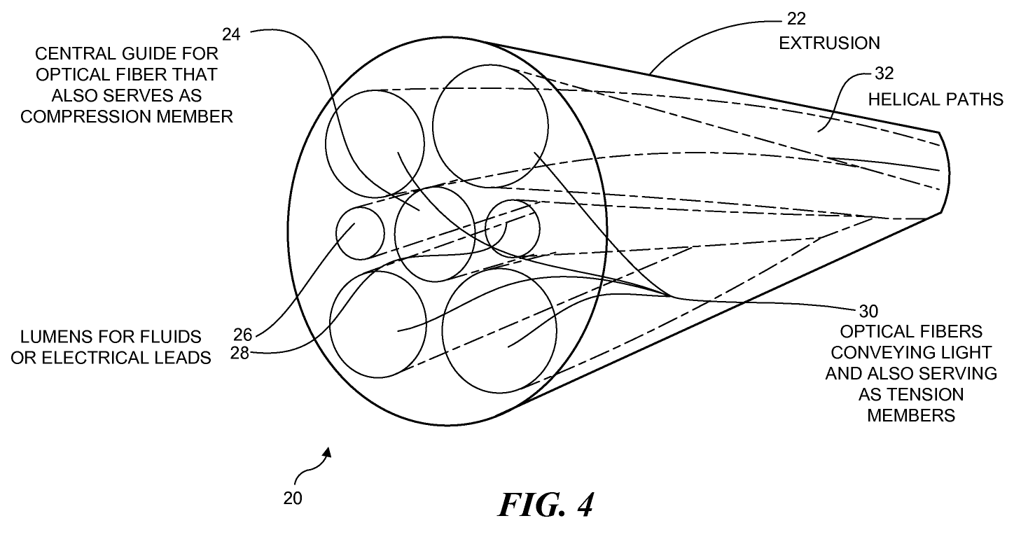

If the inner member is employed as a compression member, and the outer helical member serves as a tension member for use in bending the distal tip of a flexible endoscope, a guide will be required to retain the outer tension member in the helical path so that it can provide tension in regard to the compression member. FIG. 4 illustrates how such a helical guide 20 can be made from an extrusion 22 having a central lumen 24 that extends longitudinally down the center of the extrusion, and a plurality of lumens 26 and 28 extending longitudinally on diametrically opposite sides of the central lumen. Lumen 26 is provided to convey one of two tension members (neither shown in this Figure), and lumen 28 is provided to convey the other. The pitch of the helix of lumens 24 and 26 is about 1 cm in this exemplary embodiment.

While not shown in FIG. 4, in this exemplary embodiment, central lumen 24 is provided to guide an optical fiber between proximal and distal ends of the extrusion. The optical fiber transmits light for scanning a surface that is adjacent to the distal end of the flexible endoscope and also serves as the compression member when bending the distal end of the extrusion. It will be understood that the compression member need not comprise an optical fiber, since other materials can be employed for this purpose. However, use of an optical fiber for the central compression member has the benefit of making the best use of the cross-sectional size of the flexible extrusion and avoids the need to increase its size to accommodate a compression member that does not also carry out another required function.

The helical guide passages provided by lumens 26 and 28 should have a low coefficient of friction to reduce the loss in tension at the distal end, as tension is selectively applied to one of the optical fibers comprising the tension members to bend the distal tip of the extrusion (i.e., of the flexible endoscope). For example, polytetrafluoroethylene, an example of which is sold by DuPont as TEFLON™, has a relatively low coefficient of friction (dynamic) of about 0.1 and would be a good exemplary choice material for the flexible extrusion. Materials employed for the tension member should have properties such as high tensile strength, flexibility, toughness, and a low coefficient of friction, which are all characteristics of optical fibers. Accordingly, optical fibers can function very well as tension members in a flexible endoscope, particularly if coated with polytetrafluoroethylene or other low friction materials to ensure that they slide within lumens 26 and 28 with little friction.

In this exemplary embodiment of a flexible endoscope, the internal surface of central lumen 24 is bonded to the central optical fiber (or other type of compression member) to transfer compression loading to the member. The bonding can be continuous along most of the length of the central lumen or at spaced-apart longitudinal intervals. One exemplary adhesive suitable for bonding the optical fiber to the extrusion within the central lumen is sold by Norton Performance Plastics Corporation of Wayne, N.J. as CHEMGRIP™, although other suitable adhesives can instead be employed. To ensure a good bond with the low friction material used for the extrusion (e.g., for example, a TEFLON™ material), it may be necessary to etch the material before the bonding step is attempted.

By bonding the extrusion to the compression member in this manner, and by employing a helical path 32 (i.e., helically extending lumens) for the optical fibers comprising the tension members, the force applied to the tension members will not bend or distort a tether section of the flexible endoscope. The tether section is the portion of the flexible endoscope disposed between the distal tip and the proximal end of the flexible endoscope. Distortion of the tether section that would otherwise likely occur, as shown by the schematic illustrations in FIGS. 1A and 1B is avoided, since the helical lumens and the optical fibers comprising the tension members within them correspond to the schematic illustrations of FIGS. 2 and 3.

The extrusion also can include additional lumens 30 that extend longitudinally and helically, for providing access to the distal tip and to provide passages for one or more additional components such as more optical fibers, wires (i.e., conductors for conveying electrical signals or power), liquids, and gases. These additional components can be employed in an exemplary flexible endoscope to enable functions such as: (1) performing biopsies; (2) improving visibility, for example, by circulating liquids and/or gases to an internal site proximate to the distal end of the flexible endoscope, or by employing suction to withdraw a sample of body fluid or simply to clear body fluid from a body passage through which the flexible endoscope is being advanced or from where it is used to carry out other functions; and, (3) administering therapies, e.g., drug delivery, high intensity light delivery, etc.

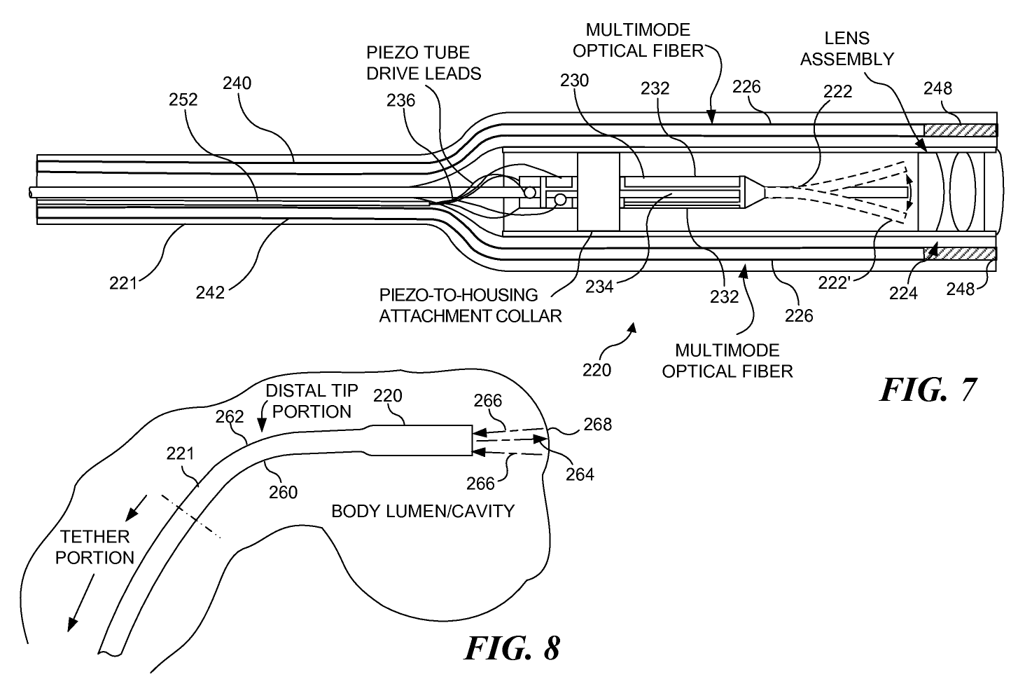

Near the distal end of the flexible endoscope, the tension members are attached within their respective lumens at an attachment point (as discussed below in connection with FIG. 7). These attachment points can be disposed as far as possible (radially) from the central compression member (thereby creating a longer radially directed moment arm around the optical fiber used for scanning (i.e., around the compression member), so that the tension from the tension member compresses the outer part of the extrusion adjacent to the tension member. With one side of the extrusion compressed, and the other opposite side relaxed, the distal tip of the endoscope will bend toward the tensioned side where the optical fiber under tension is attached to the flexible extrusion.

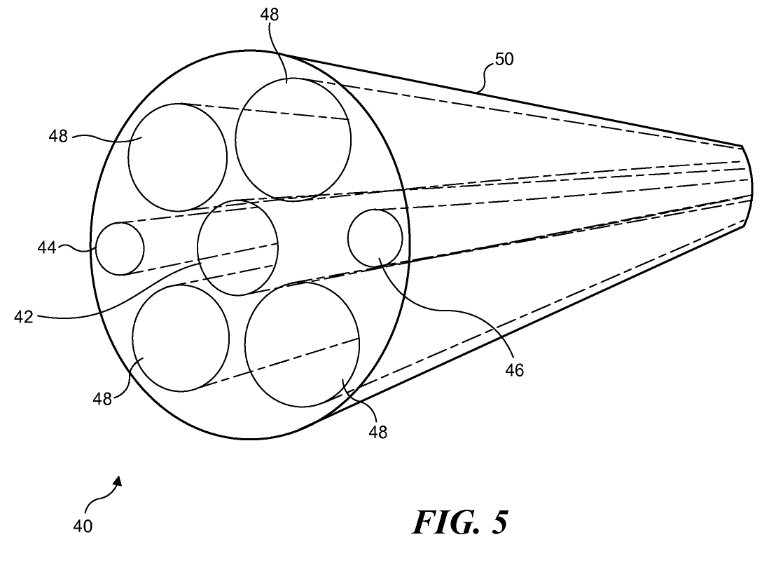

FIG. 5 shows a flexible extrusion 50 for a distal part of an exemplary flexible endoscope 40 that is actively bent using tension members (not shown in this Figure). It should be noted that lumens 44 and 46, which serve as guide passages for the optical fibers comprising the tension members, have been moved radially outward from a central lumen 42 and nearer to the perimeter of extrusion 50, compared to the position of lumens 24 and 26 in FIG. 4. Also, lumens 44 and 46 are straight and do not follow a helical path around central lumen 42. The scanning optical fiber is not bonded to the internal surface of central lumen 42 in this exemplary distal portion of extrusion 50, which enables more of the flexible extrusion to compress, thus requiring less tension in the tension member that is being pulled to bend the distal end of the extrusion. As noted above, additional lumens 48 can optionally be included in extrusion 50 to provide a path for one or more other optical fibers, wire conductors, gases, liquids, or other components the user would like to enable to be conveyed to the distal end of the extrusion, to perform various other functions, as discussed above.

The overall length of this flexible extrusion, and the tension in the optical fiber comprising the tension member, determines the radius of curvature of the distal tip bend. Making the flexible extrusion shorter increases the tension required for a given angle of deflection, but also decreases the radius of curvature. Making the extrusion long decreases the tension required for a given angle of deflection of the distal tip, but also increases the radius of curvature.

The exemplary embodiment discussed above gives the capability to bend the tip back and forth (i.e., in either of two opposite directions) in one plane or along one axis. A second plane for bending motion for the distal tip of a flexible endoscope along a second orthogonal axis can be achieved by adding a second pair of tension members that are disposed on diametrically opposite sides of the central lumen and on a line through the center of the central lumen that is rotated 90 degrees relative to a line through the central lumen and the first pair of lumens used for the first pair of tension members. The optical fibers comprising the first and second pair of tension members that extend through these four lumens thus provide a user the ability to bend the distal tip of the flexible endoscope relative to two orthogonal axes.

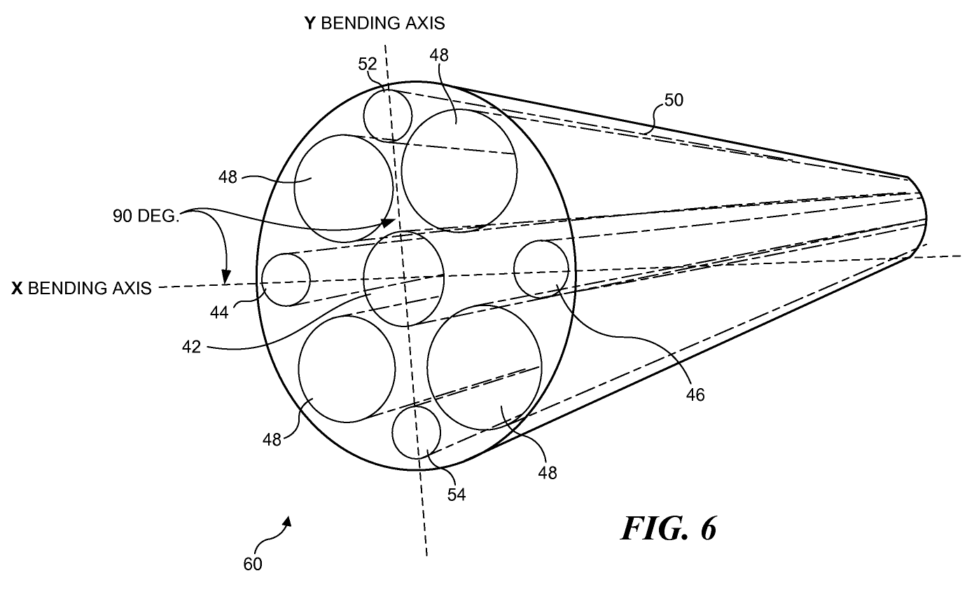

FIG. 6 shows how the distal tip of a flexible endoscope 60 appears for an exemplary embodiment that a user is able to selectively bend relative to two orthogonal axes. The helical extrusion of the tether portion of this embodiment appears similar to the single bending axis exemplary embodiment shown in FIG. 4, but has two additional helical lumens 52 and 54 that serve as guides for the second pair of tension members. By applying different tensions to these four tension members, complete bending control of the distal tip can be achieved, which is a very important capability in long, very small diameter flexible endoscopes. Present practice is to enable an endoscope distal tip to bend in only one direction, and when necessary to look in a different direction, the endoscope is physically twisted. However, when employing a very small diameter, long flexible endoscope, attempting to twist the shaft of the endoscope can generate a sufficiently large torque to damage the endoscope. Accordingly, it will generally be preferable to employ a flexible endoscope that includes two pairs of tension members disposed at cardinal points around the central lumen, so that complete bending control of the distal tip in any direction can be accomplished.

### Detailed Description of Distal End of Exemplary Flexible Endoscope

The distal end of exemplary flexible endoscope 220 shown in FIG. 7 includes a scanning optical fiber 222, which is driven to scan in a desired scan pattern at or near its resonant frequency, as indicated by its positions in phantom view, e.g., at reference numeral 222′. A lens assembly 224 is provided at the distal end of flexible endoscope 220 and is employed for focusing the light exiting the scanning optical fiber 222 onto an adjacent site. Two or more multimode optical fibers 226 are disposed peripherally around scanning optical fiber 222, within a flexible extrusion 221, and are used for conveying reflected light to one or more photodetectors (not shown) that are disposed at the proximal end of the flexible endoscope.

Scanning optical fiber 222 is driven in a desired pattern by a piezoelectric tube actuator 230 relative to two orthogonal axes, in response to drive signals supplied to electrodes 232 and 234 through electrical leads 236, which extend proximally through a lumen 252 within the flexible extrusion of flexible endoscope 220. A single-axis (linear) scan pattern can, for example, be generated by applying voltage to one electrode 232 or to opposing electrodes 234 of piezoelectric tube actuator 230. By applying an oscillating periodic voltage (e.g., a sine wave) having a frequency at or near the mechanical resonant frequency of the base-excited scanning optical fiber cantilever to the actuator through electrical leads 236, the amplitude of the tip motion can be mechanically amplified due to the mechanical resonance of the scanning optical fiber cantilever. Furthermore, for example, the concurrent application of a second periodic voltage (a cosine wave) to electrodes 234 (which are orthogonal to electrodes 232) on the actuator, at the same or slightly different resonant frequency, causes the resonating optical fiber tip to move in an elliptical scanning pattern.

A signal useful for producing an image is generated by the optical fiber scanner shown in FIG. 7, by directing the light emitted from scanning optical fiber 222 onto the surface or a region at an internal site that is adjacent to the distal end of the flexible endoscope. Light transmitted toward the region by the scanning optical fiber cantilever is focused using imaging lenses 224. Typically, the imaging lenses focus the light, directing it to a scanned portion of the internal site as the scanning optical fiber resonantly scans the site with either a linear (one-dimensional), spiral, elliptical, or other two-dimensional patterns. By varying the amplitude of the voltages applied to the actuator during the elliptical scan, a two-dimensional (2-D) space-filling scanning pattern is formed.

Proximal of piezoelectric tube actuator 230, flexible extrusion 221 includes lumens 240 and 242, which are disposed on diametrically opposite sides of the scanning optical fiber and the lumen through which it extends. Within lumens 240 and 242 are disposed multimode optical fibers 226. Multimode optical fibers 226, which thus surround the scanning optical fiber, receive the light that is reflected from tissue at the internal site, and this light, which conveyed proximally through the multimode optical fibers, is used for generating the 2-D image or for evaluating parameters of the tissue. Typically, multimode optical fibers 226 convey the received light to one or more detectors (not shown) that are disposed at the proximal end of the optical fiber scanner, and which produce signals used for imaging the site or for other purposes, such as diagnostic evaluation.

It should be emphasized that in this exemplary embodiment, scanning optical fiber 222 also serves as a compression member to facilitate bending the distal end of the flexible endoscope, as illustrated and discussed below in connection with FIG. 8. In addition, in this exemplary embodiment, multimode optical fibers 226 also serve as tension members, so that when a tension is selectively applied to one of the tension members (i.e., to one of multimode optical fibers 226), the distal end of the flexible endoscope is deflected in the direction corresponding to the side of the flexible extrusion on which the multimode optical fiber under tension is disposed and attached.

The distal ends of tension member/multimode optical fibers 226 are attached or coupled to flexible extrusion 221 by adhesive patches 248. The adhesive patches can be a thermally or chemically set adhesive, or other suitable type of adhesive. For example, a thermal adhesive may be used that melts when heated and solidifies when cooled, to anchor the distal ends of the tension members at attachment points within their respective lumens. These attachment points are directly adjacent to the distal end of flexible extrusion 221.

For purposes of clarity, FIG. 7 does not show two additional lumens and multimode optical fibers, which are optionally included in extrusion 221, to enable bending of the distal end of flexible endoscope 220 in a second plane or relative to a second axis that is orthogonal to the first plane or axis in which the illustrated tension members (i.e., multimode optical fibers 226) can bend the distal tip. Providing these second pair of lumens and second pair of tension members enables a user to selectively bend the distal tip of the flexible endoscope in any desired direction by applying appropriate tension to one or more of the four tension members having distal ends connected to the extrusion at cardinal attachment points around the scanning optical fiber, so that the distal end of the flexible endoscope can be bent relative to two orthogonal planes or axes.

In the tether portion of the flexible endoscope (not shown in this Figure) that begins a few centimeters proximal of the distal portion of flexible endoscope 220 of FIG. 7, the lumens disposed around the scanning optical fiber twist in a helical spiral, generally as shown for exemplary flexible endoscope 20 in FIG. 4. The helical twist can be formed in extrusion 221 by heating the flexible extrusion sufficiently to soften it. The tether portion of the extrusion is then twisted at its proximal end sufficiently to create the desired helical spiral of the lumens that serve as guides for the tension members (and also, to form the helical spiral of the other lumens disposed around the central lumen). The scanning optical fiber is then bonded to extrusion 221 within the tether portion of the flexible endoscope, either continuously along its length in that portion or at longitudinally spaced-apart points therein. Bonding the scanning optical fiber to the flexible extrusion thus sets and maintains the helical twist of these surrounding lumens after the flexible extrusion cools.

FIG. 8 illustrates how a flexible endoscope can be selectively bent by applying tension to a tension member comprising a multimode optical fiber 226 (that is close to an inside radius 260 of the bend, compared to another tension member comprising a multimode optical fiber that is close to an outside radius 262 of the bend), relative to the compression member comprising the scanning optical fiber. By thus bending the distal end of flexible endoscope 220, it is possible to more readily advance it through circuitous body passages to a desired position in a body lumen/cavity. Also, by bending the flexible endoscope as desired, the distal end of flexible endoscope 220 can be positioned adjacent to tissue 268 that is to be imaged (or treated with an optical therapy delivered through one or more of the optical fibers or through one or more optical fibers that are not shown). The scanning optical fiber can then emit illumination light 264 directed toward tissue 268, and the multimode optical fibers within the flexible endoscope can receive reflected light 266 from the tissue in a patient's body and convey the reflected light toward the proximal end of the flexible endoscope to produce images of the tissue that are displayed to medical personnel.

Although the concepts disclosed herein have been described in connection with the preferred form of practicing them and modifications thereto, those of ordinary skill in the art will understand that many other modifications can be made thereto within the scope of the claims that follow. Accordingly, it is not intended that the scope of these concepts in any way be limited by the above description, but instead be determined entirely by reference to the claims that follow.

The invention in which an exclusive right is claimed is defined by the following:

1. A flexible endoscope having a distal tip that can be selectively bent, comprising:
(a) a flexible extrusion that is elongate, extending between a proximal end and a distal end and having a plurality of lumens formed therein, at least two lumens of the plurality of lumens being disposed on diametrically opposite sides of, and radially outward of a central lumen within the flexible extrusion;
(b) at least a pair of tension members, each tension member comprising an optical fiber that extends proximally through a different one of the plurality of lumens from an attachment point where the optical fiber is coupled with the flexible extrusion, each attachment point being disposed adjacent to the distal tip of the flexible extrusion, the optical fibers comprising the tension members being free to slide longitudinally within the lumens proximal of the attachment points, each optical fiber comprising a tension member also being employed for conveying a light signal in the flexible endoscope; and
(c) a central compression member disposed within the central lumen that is bonded to the flexible extrusion proximal of a distal portion of the extrusion, so that when tension is applied to a first tension member comprising an optical fiber relative to the compression member, the distal tip of the flexible endoscope is bent in a first direction, and when tension is applied to a second tension member comprising an optical fiber that is disposed generally diametrically opposite the first tension member, the flexible endoscope is bent in a second direction that is opposite to the first direction, enabling the distal tip of the flexible endoscope to be selectively caused to bend in either of at least two generally opposite directions.

2. The flexible endoscope of claim 1, wherein the compression member comprises a scanning optical fiber that is also used to convey light for producing an image of a surface adjacent to the distal end of the flexible extrusion.

3. The flexible endoscope of claim 1, wherein the at least two lumens through which the tension members comprising the optical fibers extend, are disposed relatively radially closer to the central lumen in a portion of the flexible endoscope that is proximal to a distal tip portion, than in the distal tip portion of the flexible endoscope.

4. The flexible endoscope of claim 1, wherein the at least two lumens extend helically around the central lumen in a portion of the flexible endoscope that is proximal of a distal tip portion of the flexible endoscope.

5. The flexible endoscope of claim 1, wherein the flexible extrusion is formed of a material selected to have a relatively low coefficient of friction, to enable the at least the pair of tension members comprising the optical fibers to slide easily within the lumens in which the tension members are disposed.

6. The flexible endoscope of claim 5, wherein the material comprises polytetrafluoroethylene.

7. The flexible endoscope of claim 1, wherein the plurality of lumens formed in the flexible extrusion includes at least one lumen not used in connection with bending the distal tip, said at least one lumen being adapted to convey at least one element selected from the group consisting of:
(a) one or more optical fibers that are not used as the compression member or as a tension member;
(b) one or more wires;
(c) one or more liquids; and
(d) one or more gases.

8. The flexible endoscope of claim 7, wherein the at least one element that can be conveyed through the at least one lumen not used in connection with bending the distal tip, is used for at least one function selected from the group consisting of:
(a) performing a biopsy;
(b) modifying a visibility in a region of a patient's body in which the flexible endoscope is adapted to be inserted; and
(c) rendering a therapy to a portion of a patient's body.

9. The flexible endoscope of claim 1, wherein the at least the pair of lumens comprises a first pair of lumens, lumens comprising the first pair being disposed on diametrically opposite sides of the central lumen from each other, and a second pair of lumens, lumens of the second pair being disposed on diametrically opposite sides of the central lumen from each other, but lying on a plane that is generally orthogonal relative to a plane on which the first pair of lumens lie, so that a line extending through a center of the central lumen between the lumens of the first pair is generally perpendicular to a line extending through the center of the central lumen between the lumens of the second pair, and wherein the at least the pair of tension members includes a first pair of tension members comprising optical fibers, each of which is disposed in a different one of the first pair of lumens, and a second pair of tension members comprising the optical fibers, each of which is disposed in a different one of the second pair of lumens, the first pair of tension members being used to bend the distal end of the flexible endoscope relative to a first axis, and the second pair of tension members being used to bend the distal end of the flexible endoscope relative to a second axis that is generally orthogonal to the first axis.

10. The flexible endoscope of claim 1, wherein at least one optical fiber comprising a tension member conveys a light signal from a proximal end of the optical fiber to a distal end of the optical fiber.

11. The flexible endoscope of claim 1, wherein at least one optical fiber comprising a tension member conveys a light signal from a distal end of the optical fiber to a proximal end of the optical fiber.

12. A method for enabling a distal tip of a flexible endoscope to be selectively bent, comprising the steps of:
(a) providing a central lumen that extends longitudinally through a center of a flexible elongate housing, and a plurality of longitudinally extending lumens that are disposed in the housing at spaced-apart positions around the central lumen;
(b) passing a plurality of tension members comprising optical fibers that convey light through lumens comprising at least a portion of the plurality of lumens, so that the plurality of tension members are freely able to move longitudinally within the lumens through which they pass;
(c) attaching a distal end of each of the plurality of tension members to the housing at points proximate to the distal tip of the flexible endoscope; and
(d) passing a compression member through the central lumen, the compression member being mechanically connected to an inner surface of the central lumen for use in connection with any of the plurality of tension members comprising optical fibers, to which a tension force is applied, so that when a tension force is applied to one of the plurality of tension members relative to the compression member, the distal tip of the flexible endoscope is thereby caused to bend in a direction toward a point where the one tension member is attached to the housing, the compression member transferring a compression force to the housing to enable the bending of the distal tip relative to the optical fiber comprising the compression member to which the tension force is applied.

13. The method of claim 1, wherein the compression member comprises a scanning optical fiber, further comprising the step of passing light through the scanning optical fiber.

14. The method of claim 12, further comprising the step of enabling a user to apply a tension to a tension member that is attached to the housing at a point that is generally diametrically opposite the point where said one tension member is attached to the housing, thereby causing the distal tip to be bent in a different direction that is generally opposite the direction in which the distal tip is bent when the tension force is applied to said one tension member.

15. The method of claim 12, wherein each lumen in the portion includes a different one of four tension members, each tension member comprising an optical fiber that is coupled to the housing proximate the distal tip at different cardinal points around the central lumen, enabling a user to bend the distal tip in generally diametrically opposite directions for each of two orthogonal axes.

16. The method of claim 12, further comprising the step of causing the plurality of lumens to helically spiral around the central lumen to substantially reduce changes in a relative length of the tension members comprising the optical fibers that pass through the at least the portion of the plurality of lumens, when the housing is caused to bend.

17. The method of claim 12, further comprising the step of passing at least one component through at least one lumen of the plurality of the lumens not included in the portion, wherein the at least one component is selected from the group consisting of:
(a) one or more additional optical fibers that are not used as a tension member;
(b) one or more wires;
(c) one or more liquids; and
(d) one or more gases.

18. The method of claim 17, further comprising the step of enabling a user to employ the at least one component to carry out at least one function, wherein the at least one function is selected from the group consisting of:
(a) performing a biopsy;
(b) improving visibility at a site within a patient's body at which the distal tip of the flexible endoscope is disposed; and
(c) rendering a therapy to a site within a patient's body.

19. The method of claim 12, further comprising the step of providing a smaller spacing between lumens disposed on diametrically opposite sides of the central lumen through which the tension members comprising optical fibers pass, in a part of the housing that is disposed between a proximal end of the housing and the distal tip, than a corresponding spacing in a distal tip portion of the housing.

20. The method of claim 12, further comprising the step of employing a material for the housing that has a relatively low coefficient of friction to ensure that each of the plurality of tension members comprising the optical fibers can slide freely within the lumen through which the tension member passes.

21. A flexible endoscope having a distal tip that can be selectively caused to bend in different directions, comprising:
(a) a flexible extrusion that is elongate, extending between a proximal end and a distal end and having:
(i) a central lumen that extends longitudinally through a center of the flexible extrusion; and
(ii) a plurality of helical lumens formed within the flexible extrusion, spaced-apart from and around the central lumen;
(b) at least a pair of tension members, each tension member comprising an optical fiber that extends proximally through a different one of the plurality of helical lumens and is connected to the flexible extrusion at an attachment point, each attachment point being disposed adjacent to the distal tip of the flexible extrusion, the optical fibers comprising the tension members each being capable of conveying light and being free to slide longitudinally within the helical lumens proximal of the attachment points; and
(c) a compression member disposed within the central lumen and connected to the flexible extrusion within the central lumen, so that when tension is applied to an optical fiber comprising a first tension member relative to the compression member, the distal tip of the flexible endoscope is bent in a first direction, and when tension is applied to another optical fiber comprising a second tension member that is disposed generally diametrically opposite the first tension member, the flexible endoscope is bent in a second direction that is opposite to the first direction, enabling the distal tip of the flexible endoscope to be selectively caused to bend in at least two opposite directions.

22. The flexible endoscope of claim 21, wherein the at least the pair of helical lumens comprises a first pair of helical lumens disposed on opposite sides of the central lumen, and a second pair of helical lumens disposed on opposite sides of the central lumen, so that the helical lumens are spaced apart by about 90 degrees, and wherein the at least the pair of tension members include a first pair of tension members comprising optical fibers, each of which is disposed in a different one of the first pair of helical lumens, and a second pair of tension members comprising optical fibers, each of which is disposed in a different one of the second pair of helical lumens, the first pair of tension members being used to bend the distal end of the flexible endoscope relative to a first axis, and the second pair of tension members being used to bend the distal end of the flexible endoscope relative to a second axis that is generally orthogonal to the first axis.

23. The flexible endoscope of claim 21, wherein tension members disposed on opposite sides of the central lumen are spaced diametrically further apart at the distal tip, than in a tether portion of the flexible endoscope that extends from just behind the distal tip to the proximal end of the flexible endoscope.

24. The flexible endoscope of claim 21, wherein the flexible extrusion is formed of a material selected for a characteristic low coefficient of friction, so that each optical fiber comprising one of the tension members is able to readily slide within the helical lumen through which the tension member passes.

25. The flexible endoscope of claim 24, wherein the material comprises polytetrafluoroethylene.

26. A flexible endoscope having a distal tip that can be selectively caused to bend in different directions, comprising:
(a) a flexible extrusion that is elongate, extending between a proximal end and a distal end and having a plurality of lumens formed therein, at least two lumens of the plurality of lumens being disposed on opposite sides of, and radially outward of a central lumen within the flexible extrusion;
(b) at least a pair of tension members, each tension member comprising an optical fiber, and each optical fiber comprising a tension member extending proximally through a different one of the plurality of lumens from an attachment point where the optical fiber is connected to the flexible extrusion, each attachment point being disposed adjacent to the distal tip of the flexible extrusion; and
(c) a compression member disposed within the central lumen and bonded to the flexible extrusion, so that when tension is applied to a first tension member relative to the compression member, the distal tip of the flexible endoscope is bent in a first direction, and when tension is applied to a second tension member that is disposed on an opposite side of the compression member from the first tension member, the flexible endoscope is bent in a second direction that is opposite to the first direction, enabling the distal tip of the flexible endoscope to be selectively caused to bend in at least two opposite directions.

27. The flexible endoscope of claim 26, wherein the compression member comprises a scanning optical fiber that also conveys light through the central lumen, between distal and proximal ends of the flexible endoscope, and which is used for producing images of a surface adjacent to the distal end of the flexible endoscope.

28. The flexible endoscope of claim 26, wherein the plurality of lumens form a helix around the central lumen, so that when the distal tip of the flexible endoscope is caused to bend, relative lengths of the optical fibers comprising the tension members on an inner side of a bend in the distal tip and on an outer side of the bend, generally do not change.

29. The flexible endoscope of claim 26, wherein at least one component other than a tension member is conveyed through at least one of the plurality of lumens, the at least one component being used to carry out at least one function other than causing the distal tip to bend.

30. The flexible endoscope of claim 29, wherein the at least one different function comprises one or more functions selected from the group consisting of:
(a) performing a biopsy;
(b) improving visibility at a site within a patient's body at which the distal tip of the flexible endoscope is disposed; and
(c) rendering a therapy to a site within a patient's body.

31. The flexible endoscope of claim 26, wherein the flexible extrusion is formed of a polytetrafluoroethylene material having a characteristic low coefficient of friction, so that each optical fiber comprising a tension member is able to readily slide within the lumen through which the tension member extends.
